(19) United States
(12) Patent Application Publication
(10) Pub. No.: US 2026/0258365 A1
(43) Pub. Date: Sep. 3, 2026

(54) SAFE AND EFFICIENT METHOD FOR HYPOXIA-INDUCED ADIPOGENIC DIFFERENTIATION OF CELLS AND USE OF METHOD

(71) Applicant: NANJING JOES FUTURE FOOD TECHNOLOGY CO., LTD, Nanjing (CN)

(72) Inventors: Guanghong ZHOU, Nanjing (CN); Shijie DING, Nanjing (CN); Haozhe ZHU, Nanjing (CN); Chen WANG, Nanjing (CN); Yi KUANG, Nanjing (CN)

(21) Appl. No.: 19/656,122

(22) Filed: Apr. 23, 2026

(30) Foreign Application Priority Data
May 28, 2025 (CN) 202510698572.X

(51) Int. Cl. C12N 5/077; A23L 13/00

(52) U.S. Cl. CPC C12N 5/0653; A23L 13/00; C12N 2500/02; C12N 2506/13; C12N 2521/00; C12N 2527/00

(57) ABSTRACT

The present invention discloses a safe and efficient method for hypoxia-induced adipogenic differentiation of cells and use thereof, belonging to the technical field of cell culture. The method involves expanding cells to a certain number in a reactor in a suspension environment, and inducing adipogenic differentiation of the cells in suspension by reducing a dissolved oxygen level to hypoxia. The method induces adipogenic differentiation of cells solely through physical factors. Compared to traditional chemical induction adipogenic differentiation methods, no exogenous reagents are introduced during a differentiation process, and food safety is ensured. By using the method, cell adipogenic differentiation can be completed in 4-6 days, and expansion and adipogenic differentiation of cells can be completed in about 10 days, thus accelerating differentiation. Moreover, adipogenic differentiation can be completed in a large-scale reactor of, for example, 200 L, and the method can be applied to large-scale preparation of cell-cultured meat.

### TECHNICAL FIELD

The present application belongs to the technical field of cell culture and specifically relates to a safe and efficient method for hypoxia-induced adipogenic differentiation of cells and use thereof.

### BACKGROUND

Cultured meat, also known as cell-cultured meat, is produced by a technique for producing edible meat by culturing animal cells in vitro. Compared with traditional animal husbandry production methods, the nutritional composition of cell-cultured meat can be adjusted according to demand to meet health requirements of consumers, reduce environmental pollution, improve animal welfare, lower disease risks, and the like. The main components of traditional meat include skeletal muscles, fats, and connective tissues (mainly composed of collagen). In a production process of cultured meat, seed cells can achieve functional differentiation under specific induction conditions after undergoing large-scale expansion. The differentiation of adipose cells plays a crucial role in the texture, flavor, and nutritional content of meat.

There are many studies on methods for inducing adipogenic differentiation of cells in vitro, and chemical induction is a common adipogenic induction method. Experimental studies have shown that by regulating a drug combination in a culture system, cells can be effectively induced to differentiate into the adipocyte lineage. Classic drugs include phosphodiesterase inhibitors (isobutylmethylxanthine, IBMX), dexamethasone, insulin, rosiglitazone, indomethacin, and the like. Transcription factors such as PPARγ are activated through drug combinations to promote adipogenic differentiation of cells. Although these chemical induction differentiation methods are effective, the methods involve food safety issues and the time for adipogenic differentiation is usually 10 days or more, which is not conducive to industrial production. With rapid development of the cell-cultured meat industry, it has become particularly important to find a more natural, safe, and efficient method for adipogenic differentiation.

In the current technological path, seed cells of cultured meat can be adhered to a decellularized plant scaffold (e.g., sterilized tissues of onions and mushrooms) which serves as a biocompatible material to produce a lipid product, or be produced into suspended lipid globules by a self-organization technique. However, the above methods are all based on chemical inducers, and both the compatible material and the self-organization technique have difficulties in large scale production. Currently, there is no technique that optimizes adipogenic differentiation of cells solely through physical factors.

### SUMMARY

### 1. Problems to be Solved

The present application provides a safe and efficient method for hypoxia-induced adipogenic differentiation of cells and use thereof, in response to a conflict between adipogenic differentiation of cells induced by chemical substances and food safety supervision in the prior art. The method involves adjusting a dissolved oxygen concentration in a cell culture system, and specifically reducing the dissolved oxygen in the culture system after cell expansion to induce adipogenic differentiation of cells. The method can effectively improve the efficiency and maturity of adipogenic differentiation of cells, overcome limitations of traditional drug induction methods, and can be used for preparation of cell-cultured meat.

### 2. Technical Solution

In order to solve the above problems, the technical solution adopted in the present application is as follows:

The present application provides a safe and efficient method for hypoxia-induced adipogenic differentiation of cells, the method including the following steps:
S1: culturing cells using a culture medium for cell expansion, with dissolved oxygen controlled at no less than 35% during the culture process, where this stage is mainly a cell expansion stage, during which the number of cells increases to obtain a sufficient number of cells; and
S2: after the cell expansion, controlling the dissolved oxygen at no greater than 20% and continuing culturing to induce adipogenic differentiation of the cells, where at this stage, the cells do not stop expanding, but mainly undergo hypoxia-induced adipogenic differentiation, resulting in an increase in intracellular lipid droplets.

Further, the cells are a pig muscle stem cell line adapted to carrier-free and serum-free suspension culture, and the pig muscle stem cell line may be suspended and expanded, and complete adipogenic differentiation in a suspended state.

Further, the pig muscle stem cell line adapted for carrier-free and serum-free suspension culture, named piglet muscle stem cell line YP-S4-S-SC, is deposited at the China Center for Type Culture Collection, located at Wuhan University in Wuhan, China, with a deposit number of CCTCC NO: C2022372 on Dec. 7, 2022. Reference is made to the Chinese invention patent with a publication number CN116555171A for details.

Further, the culture medium is a serum-free culture medium.

Further, in S1, the dissolved oxygen is controlled at no less than 36%.

Further, in S1, the dissolved oxygen is controlled at no less than 37%.

Further, in S1, the dissolved oxygen is controlled at no less than 38%.

Further, in S1, the dissolved oxygen is controlled at no less than 39%.

Further, in S1, the dissolved oxygen is controlled at no less than 40%.

Further, in S1, the dissolved oxygen at the initial stage of culture is controlled at 90-100%.

Further, in S2, the dissolved oxygen is controlled at 1-20%.

Further, in S2, the dissolved oxygen is controlled at 9-20%.

Further, in S2, the dissolved oxygen is controlled at 9-15%.

Further, in S1, the culture time is 3-10 days.

Further, in S1, the culture time is 4-8 days.

Further, in S1, the culture time is 5-6 days.

Further, in S2, the culture time is 3-10 days.

Further, in S2, the culture time is 4-8 days.

Further, in S2, the culture time is 5-6 days.

Further, in the safe and efficient method for hypoxia-induced adipogenic differentiation of cells, a culture process is a fed-batch culture process, a serum-free feed culture medium is supplemented from D3 of culture, and the feed culture medium is supplemented every 2 days thereafter.

Further, in the fed-batch culture process, the volume of the feed culture medium may be adjusted based on a density of live cells, a cell viability, and concentrations of glucose, lactic acid, ammonium ions, and amino acid detected by sampling. As an example, in the present application, a serum-free feed culture medium is supplemented at 10% of the culture volume.

Further, S1 includes:
seeding cells harvested from cell thawing into a stirred bioreactor containing a serum-free culture medium at a density of (1-10)×10⁵ cells/mL, setting a stirring speed to 80-240 rpm, a pH value to (6.0-8.0)±0.2, and a temperature to 34-40° C., setting a minimum dissolved oxygen, supplementing a serum-free feed culture medium from D3 of culture, and supplementing the serum-free feed culture medium every 2 days thereafter.

Further, S1 includes:

seeding cells harvested from cell thawing into a bioreactor containing a serum-free culture medium at a density of (2-5)×10⁵ cells/mL, and setting a stirring speed to 120 rpm, a pH value to 7.2±0.2, and a temperature to 37° C.

Further, in the safe and efficient method for hypoxia-induced adipogenic differentiation of cells, the culture process is a perfusion culture process, and perfusion culture is started at a rate of 0.5 VVD from D3 of culture, then at a rate of 1 VVD on D4, and at a rate of 2 VVD from D5.

Further, S1 includes:

seeding cells harvested from cell thawing into a wave bioreactor containing a serum-free culture medium at a density of (1-10)×10⁵ cells/mL, setting an angle of the wave bioreactor to 6-9°, a swing speed to 15-30 rpm, a pH value to (7.0-7.6)±0.2, and a temperature to 34-40° C., setting a minimum dissolved oxygen, and starting perfusion culture from D3 of culture.

Further, S1 includes:

seeding cells harvested from cell thawing into a wave bioreactor at a density of (2-5)×10⁵ cells/mL, setting an angle of the wave bioreactor to 7°, a swing speed to 20 rpm, a pH value to 7.2±0.2, and a temperature to 37° C.

Further, in the safe and efficient method for hypoxia-induced adipogenic differentiation of cells, cell thawing is performed before S1, including:

seeding cells in a serum-free culture medium at a density of (1-10)×10⁵ cells/mL, collecting the cells by centrifugation every 2-6 days for passage, repeating to obtain a sufficient number of cells, and harvesting the cells by centrifugation.

Further, the cell thawing is performed by seeding cells in a serum-free culture medium at a density of (2-5)×10⁵ cells/mL, and collecting the cells by centrifugation every 3 days for passage.

The present application further provides use of the safe and efficient method for hypoxia-induced adipogenic differentiation of cells in preparation of cell-cultured meat, specifically, for inducing adipogenesis in cells.

### 3. Beneficial Effects

Compared with the prior art, the present application has the following beneficial effects:

(1) The present application provides the safe and efficient method for hypoxia-induced adipogenic differentiation of cells and the use thereof. The method involves expanding cells to a certain number in a reactor in a suspension environment, and inducing adipogenic differentiation of the cells in suspension by reducing a dissolved oxygen level to hypoxia. The present application induces adipogenic differentiation of cells solely through physical factors. Compared to traditional chemical induction adipogenic differentiation methods, no exogenous reagents are introduced during a differentiation process, and food safety is ensured. By using the method, cell adipogenic differentiation can be completed in 4-6 days, and expansion and adipogenic differentiation of cells can be completed in 10 days, thus shortening the differentiation time. Moreover, adipogenic differentiation can be completed in a large-scale reactor of, for example, 200 L, and the method can be applied to large-scale preparation of cell-cultured meat.

(2) The safe and efficient method for hypoxia-induced adipogenic differentiation of cells and the use thereof provided by the present application particularly use the perfusion culture process for cell expansion and differentiation, and maintain a high expansion rate during the process of cell differentiation.

### BRIEF DESCRIPTION OF THE DRAWINGS

FIG. 1 shows changes of a dissolved oxygen level in a 1 L stirred reactor in Example 1.

FIG. 2 shows a cell growth curve during a hypoxia-induced adipogenic differentiation process of cells in suspension based on a fed-batch process in Example 1.

FIG. 3 shows oil red O staining images of lipid droplets obtained by the hypoxia-induced adipogenic differentiation of cells in suspension (hypoxia differentiation) and differentiation without hypoxia treatment (control), with a unit size representing 50 μm.

FIG. 4 shows changes of a dissolved oxygen level in a 1 L stirred reactor in Comparative Example 1.

FIG. 5 shows changes of a dissolved oxygen level in a 200 L stirred reactor in Example 2.

FIG. 6 shows a cell growth curve during a hypoxia-induced adipogenic differentiation process of cells in suspension based on a fed-batch process (200 L pilot scale) in Example 2.

FIG. 7 shows oil red O staining images of lipid droplets obtained by the hypoxia-induced adipogenic differentiation of cells in suspension in Example 2, with a unit size representing 50 μm.

FIG. 8 shows changes of a dissolved oxygen level in a 50 L wave reactor in Example 3.

FIG. 9 shows a cell growth curve during a hypoxia-induced adipogenic differentiation process of cells in suspension based on a perfusion culture process in Example 3.

FIG. 10 shows oil red O staining images of lipid droplets obtained by the hypoxia-induced adipogenic differentiation of cells in suspension in Example 3.

FIG. 11 shows a photo of cell cultured pork meatballs made from a cell raw material of hypoxia-induced adipogenic differentiation obtained in the present application.

FIG. 12 shows detection results of the expression levels of a hypoxia-inducible factor HIF-1α and an adipogenic factor PPARγ in the hypoxia-induced adipogenic differentiation of cells in suspension (hypoxia) and differentiation without hypoxia treatment (control).

### DETAILED DESCRIPTION

The present application is further described below with reference to specific examples.

Unless otherwise defined, all technical and scientific terms used herein have the same meanings as those commonly understood by those skilled in the art belonging to the technical field of the present application. The term “and/or” used herein includes any and all combinations of one or more related listed items.

If specific conditions are not specified in the examples, conventional conditions or conditions recommended by manufacturers are followed. Reagents or instruments used without specifying the manufacturers are conventional products that can be commercially available.

Concentration, quantity, and other numerical data may be presented in a range format herein. It should be understood that such a range format is used merely for convenience and brevity, and should be flexibly interpreted as not only including numerical values explicitly stated as limits of the range, but also including all individual numerical values or sub-ranges within the range, as if each numerical value and sub-range is explicitly stated. For example, a numerical range of about 1 to about 4.5 should be interpreted as not only including the that only describe one numerical value, such as “less than about 4.5”, which should be interpreted as including all of the aforementioned values and ranges. Furthermore, regardless of the breadth of the scope or features described, this interpretation should apply. Those skilled in the art know that “about” and “around” indicate numerical values within a certain range that can achieve the same effect.

In the present application, unless otherwise stated, the cells are the pig muscle stem cell line adapted for carrier-free and serum-free suspension culture, which is domesticated by the applicant, named piglet muscle stem cell line YP-S4-S-SC, and is deposited at the China Center for Type Culture Collection, located at Wuhan University in Wuhan, China, with a deposit number of CCTCC NO: C2022372 on Dec. 7, 2022. Reference is made to the Chinese invention patent with a publication number CN116555171A for details.

In the present application, unless otherwise specified, the serum-free culture medium used for culture is a culture medium with clear chemical composition for in-vitro proliferation of myogenic cells, includes cell culture supplementary factors and a cell proliferation culture medium free of serum components, and specifically, is a complete cell proliferation culture medium with clear chemical composition in Group 14 in Example 1 of the Chinese invention patent with a publication number CN114574433A. The serum-free feed culture medium is also a serum-free culture medium. Specifically, the cell proliferation culture medium is a 99 vol % DMEMIF12 basal culture medium supplemented with a 1 vol % penicillin-streptomycin dual antibody. In the penicillin-streptomycin dual antibody, the content of penicillin is 10,000 U/mL, and the content of streptomycin is 10 mg/mL. The types and concentrations of the cell culture supplementary factors are shown in Table 6:

TABLE 6
Types and concentrations of cell culture supplementary factors

| Component | Name | Concentration | |
|---|---|---|---|
| Component 1 | Tween 80 | 20 | μg/mL |
| Component 2 | Arachidonic acid | 10 | ng/mL |
| Component 3 | Cholesterol | 2 | μg/mL |
| Component 4 | Vitamin E acetate | 1 | μg/mL |
| Component 5 | Linoleic acid | 100 | ng/mL |
| Component 6 | Linolenic acid | 100 | ng/mL |
| Component 7 | Myristic acid | 100 | ng/mL |
| Component 8 | Oleic acid | 100 | ng/mL |
| Component 9 | Palmitic acid | 100 | ng/mL |
| Component 10 | Palmitoleic acid | 100 | ng/mL |
| Component 11 | Pluronic F68 | 1000 | μg/mL |
| Component 12 | Stearic acid | 100 | ng/mL |
| Component 13 | Insulin | 0.05 | μg/mL |
| Component 14 | Transferrin | 20 | μg/mL |
| Component 15 | Cholamine | 20 | μg/mL |
| Component 16 | Sodium selenite | 0.1 | μg/mL |
| Component 17 | Dexamethasone | 1.5 | ng/mL |
| Component 18 | Albumin | 0.5 | mg/mL |
| Component 19 | Insulin-like growth factor | 0.05 | μg/mL |
| Component 20 | Epidermal growth factor | 0.05 | μg/mL |
| Component 21 | Basic fibroblast growth factor | 0.05 | μg/mL |
| Component 22 | Leukemia inhibitory factor | 0.1 | μg/mL |
| Component 23 | Ascorbic acid | 50 | μg/mL |
| Component 24 | Sodium ascorbyl phosphate | 50 | μg/mL |
| Component 25 | Y-27632 2HCI (Rock inhibitor) | 30 | μg/mL |
| Component 26 | N-2-Hydroxyethylpiperazine-N-2- | 200 | μg/mL |
| | ethanesulfonic acid | | |
| Component 27 | Cortisol | 3 | ng/mL |
| Component 28 | Water-soluble vitamin E | 7.5 | μg/mL |
| Component 29 | Forskolin | 1 | ng/mL |
| Component 30 | Hepatocyte growth factor | 30 | ng/mL |
| Component 31 | N-acetyl-L-cysteine | 200 | μg/mL |
| Component 32 | Sodium oleoyl lysophosphatidate | 200 | μg/mL |
| Component 33 | Non-essential amino acid supplement | 79.6 | μg/mL |

In the present application, unless otherwise specified, dissolved oxygen, also known as oxygen dissolved, refers to the content of molecular oxygen dissolved in water, which is usually affected by temperature, salinity, and biological activity in water, and is usually measured in mg/L. In the present application, 10000 dissolved oxygen is a saturated dissolved oxygen level of a culture medium (aqueous phase) at 37° C., and is about 6.6 mg/L.

As used in the present application, Volume to Volume Dilution (VVD) of a culture medium refers to the ratio of the volume of a fresh culture medium added to the culture system per unit time to the total volume of the culture system, usually expressed as a volume ratio per hour in units of h⁻¹.

In the present application, unless otherwise specified, cell density refers to the number of cells contained in a unit volume (such as per milliliter) of cell suspension, and reflects the density of cells in the culture system.

In the present application, unless otherwise specified, cell viability refers to the proportion of live cells in the total number of cells, usually expressed as a percentage, and is an important indicator for measuring the health status of cells and the suitability of culture conditions.

In the present application, unless otherwise specified, intracellular lipid droplets are observed and analyzed through oil red O staining detection of lipid droplets. Oil red O is a lipid-soluble azo stain, the chemical structure of which allows oil red O to specifically bind to lipid substances. When encountering intracellular lipid droplets, due to the fact that the lipid droplets are mainly composed of neutral fats (such as triglycerides) and other lipid components, oil red O molecules are soluble in the lipid droplets, causing the lipid droplets to be stained red. Based on the principle “like dissolves like”, oil red O preferentially interacts with lipid components in intracellular lipid droplets, rather than binding with other intracellular water-soluble components. Such specificity enables clear differentiation of lipid droplets from other intracellular structures under a microscope, enabling localization and observation of lipid droplets. The oil red O staining detection of lipid droplets includes the following steps: Cell counting is performed, and based on the cell counting results, a cell suspension of 3×10⁶ cells is taken into a 1.5 mL EP tube. The EP tube is placed in a centrifuge for centrifugation at 330×g for 5 min, and the supernatant is removed. The cells are resuspended in 500 L of 4% paraformaldehyde and the cells are fixed at room temperature for 30 min or overnight at 4° C. The fixed cells are centrifuged at 330×g for 5 min, the supernatant is removed, the cells are resuspended and washed with 500 L of PBS, and centrifugation is performed again under the same conditions to remove the supernatant. 200 L of a detergent in an Oil Red O staining kit (purchased from Shanghai Beyotime Biotechnology Co., Ltd., item number: CO158M) is slowly added along the inner wall of a centrifuge tube to cover the surface of cell pellet. During liquid addition, the cells are prevented from being blown up. The detergent is pipetted after 20 s, 200 L of oil red O stain is added, the cells are blown off, and the cells are stained in the dark for 10 min. After staining, centrifugation is performed at 330×g for 5 min to remove the supernatant, and the detergent is added to cover the surface of the cell pellet. The detergent is pipetted after 20 s and the cells are resuspended in 200 L of PBS. 20 L of the resuspended cells are pipetted and added to a 96 well plate. Then, an inverted light microscope is prepared and microscope software is run, the cells are placed under the microscope, and photos are taken at 400×.

In the present application, the 1 L stirred reactor was purchased from Applitech Biological Technology Co., Ltd., with the model of my-Control. The stirred reactor maintains the dissolved oxygen in the reactor at around the minimum dissolved oxygen level by setting the minimum dissolved oxygen parameters.

In the present application, the 200 L stirred reactor was purchased from Shanghai Duoning Biological Technology Co., Ltd., with the model of DuoBioX® Pro. The stirred reactor maintains the dissolved oxygen in the reactor at around the minimum dissolved oxygen level by setting the minimum dissolved oxygen parameters.

In the present application, the 50 L wave reactor was purchased from Wuhan CEKG Technology Co., Ltd., with the model of SKC600. The wave reactor maintains the dissolved oxygen in the reactor at around the minimum dissolved oxygen level by setting the minimum dissolved oxygen parameters.

### Example 1

This example provides a safe and efficient method for hypoxia-induced adipogenic differentiation of cells, which is specifically a method for hypoxia-induced adipogenic differentiation of cells in suspension.

The method for hypoxia-induced adipogenic differentiation of cells in suspension includes the following steps:

### S1: Expansion of Cells in a Shake Flask

A stem cell line YP-S4-S-SC was thawed using a serum-free culture medium, and seeded into a 125 mL shake flask containing a serum-free culture medium at a density of 2×10⁵ cells/mL. The temperature was set to 37° C. and the rotation speed was set to 120 rpm. The cells were collected by centrifugation at 500×g for 5 min on D3.

### S2: Expansion of Cells in a Reactor

The cells harvested from S1 were seeded into a 1 L stirred reactor containing a serum-free culture medium at a density of 2×10⁵ cells/mL. The stirring speed of the stirred reactor was set to 120 rpm, the pH was set to 7.2±0.2, the temperature was set to 37° C., and the minimum dissolved oxygen was set to 40%.

A serum-free feed culture medium was supplemented at 10% of the culture volume from D3 of culture, and the serum-free feed culture medium was supplemented at 10% of the culture volume every 2 days thereafter.

### S3: Differentiation of Cells in a Reactor

After 5 days of expansion of cells in a reactor, the minimum dissolved oxygen in the stirred reactor was adjusted to 10% to perform hypoxia-induced adipogenic differentiation of cells in suspension. The serum-free feed culture medium was continuously supplemented at 10% of the culture volume every 2 days, and differentiation of the cells in suspension was completed after 5 days of continuous culture.

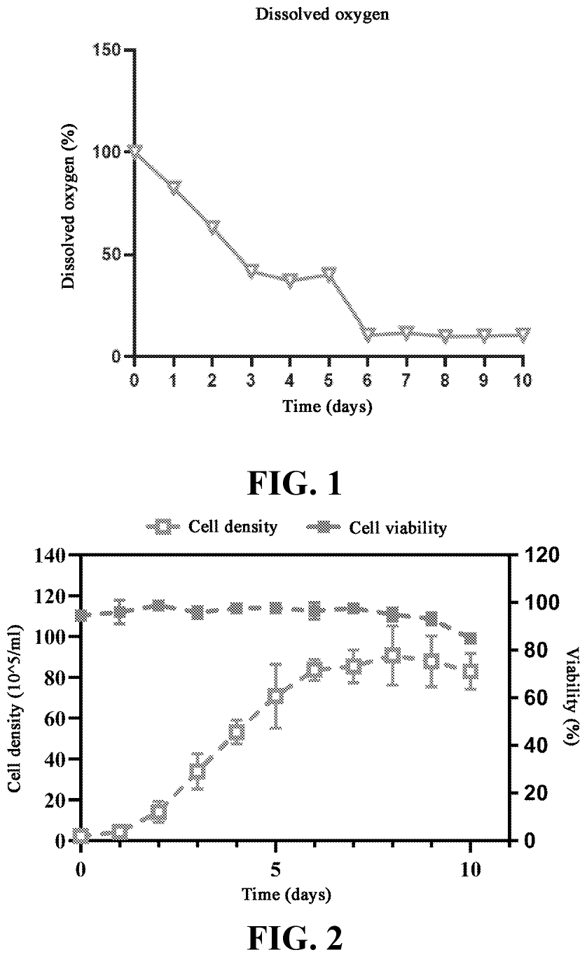

During an expansion culture period of the cells in the reactor, the dissolved oxygen in the stirred reactor was tracked and detected. As shown in Table 1 and FIG. 1, the dissolved oxygen in the cell reactor gradually decreased from 98% to around 40% as the cells expanded. This was because the minimum dissolved oxygen was set at 40%, and the cell reactor would replenish oxygen to maintain the dissolved oxygen at around 40%. On D6, due to the minimum dissolved oxygen set at 10%, the dissolved oxygen in the cell reactor rapidly decreased to around 10%.

TABLE 1

| Days | D 0 | D 1 | D 2 | D 3 | D 4 | D 5 | D 6 | D 7 | D 8 | D 9 | D 10 |
|---|---|---|---|---|---|---|---|---|---|---|---|
| Dissolved oxygen (%) | 98.0 | 82.5 | 63.2 | 41.8 | 37.2 | 40.2 | 10.7 | 11.6 | 10 | 10.3 | 10.6 |

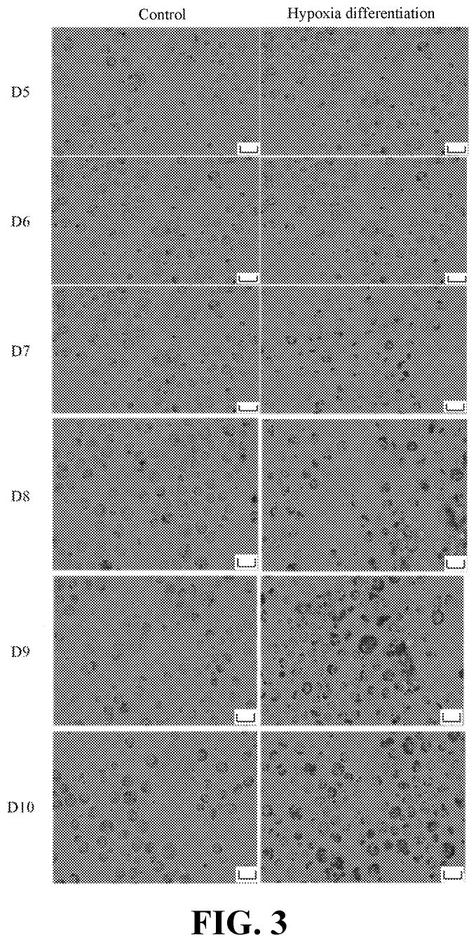

During an expansion culture period of the cells in the reactor, the corresponding growth curve was obtained by counting daily. The growth curve is shown in FIG. 2, indicating that the cell growth was in good condition and approached the peak density on D5.

During a differentiation culture period of the cells in the reactor, the corresponding growth curve was obtained by counting daily, and oil red O staining detection was performed on lipid droplets. The growth curve is shown in FIG. 2, and hypoxia differentiation began after the peak density was approached on D5. During the cell differentiation process, the number of cells could still be maintained, and the viability remained at 80% or higher all the time.

The oil red O staining results of the lipid droplets after hypoxia-induced cell differentiation are shown in the hypoxia differentiation group in FIG. 3. A small amount of lipid droplets appeared in the cells on D7, and the intracellular lipid droplets became larger and increased from D8 to D10. A large number of cells forming multilocular lipid droplets were observed on D10.

### Comparative Example 1

This comparative example provides a method for inducing adipogenic differentiation of cells in suspension.

Referring to Example 1, the difference of Comparative Example 1 is that after 5 days of cell expansion, the minimum dissolved oxygen was not adjusted, that is, the minimum dissolved oxygen was maintained at 40%, 10% of serum-free feed culture medium was continuously supplemented every 2 days, and differentiation of the cells in suspension was completed after 5 days of continuous culture.

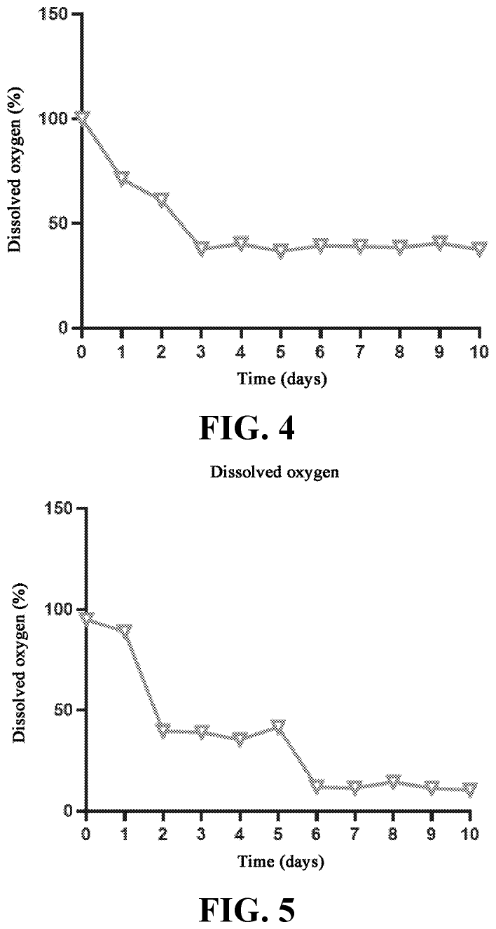

During an expansion culture period of the cells in the reactor, the dissolved oxygen in the stirred reactor was tracked and detected. As shown in Table 2 and FIG. 4, the dissolved oxygen in the cell reactor gradually decreased from 95% to around 40% as the cells expanded. This was because the minimum dissolved oxygen was set at 40%, and the cell reactor would replenish oxygen to maintain the dissolved oxygen in the reactor at around 40% all the time.

TABLE 2

| Days | D 0 | D 1 | D 2 | D 3 | D 4 | D 5 | D 6 | D 7 | D 8 | D 9 | D 10 |
|---|---|---|---|---|---|---|---|---|---|---|---|
| Dissolved oxygen (%) | 95.0 | 71.4 | 61.1 | 37.9 | 40.2 | 36.7 | 39.4 | 39 | 38.5 | 40.5 | 37.6 |

During a differentiation culture period of the cells in the reactor, oil red O staining detection was performed on lipid droplets daily. The oil red O staining results of the lipid droplets are shown as the control group in FIG. 3, and no significant accumulation of lipid droplets was observed in the cells during the culture process from D5 to D10.

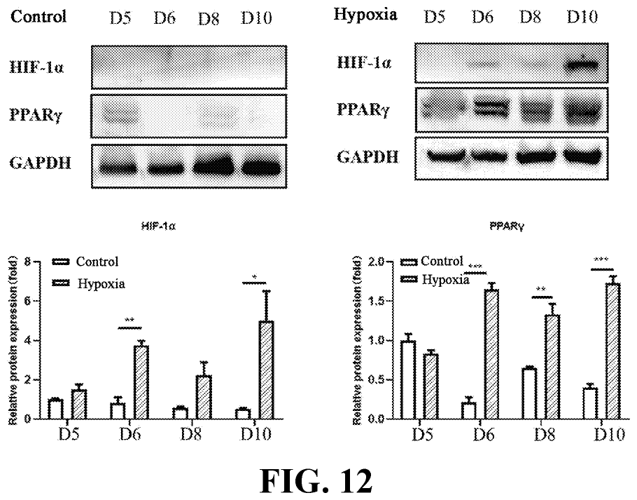

The expression levels of a hypoxia-inducible factor HIF-1α and an adipogenic factor PPARγ in the cells in Example 1 during differentiation culture (hypoxia) and the cells in Comparative Example 1 during differentiation culture (control) were detected by Western blot. As shown in FIG. 12, the expression levels of the hypoxia-inducible factor HIF-1α and the adipogenic factor PPARγ significantly increased in a hypoxic environment, indicating that the hypoxic environment upregulates expression of the key adipogenic transcription factor PPARγ via mediation of the hypoxia-inducible factor HIF-1α, thereby effectively promoting accumulation of the lipid droplets in the suspended cells.

### Example 2

This example provides a safe and efficient method for hypoxia-induced adipogenic differentiation of cells, which is specifically a method for hypoxia-induced adipogenic differentiation of cells in suspension.

In the method for hypoxia-induced adipogenic differentiation of cells in suspension, referring to Example 1, in this example, the expansion and differentiation of cells in the reactor are at a pilot scale.

The method specifically includes the following steps:

### S1: Expansion of Cells in a Shake Flask

A stem cell line YP-S4-S-SC was thawed using a serum-free culture medium, and seeded into a 125 mL shake flask containing a serum-free culture medium at a density of 2×10⁵ cells/mL. The temperature was set to 37° C. and the rotation speed was set to 120 rpm. The cells were collected by centrifugation at 500×g for 5 min on D3. The cells were seeded into a 1000 mL shake flask at a density of 2×10⁵ cells/mL, and collected by centrifugation on D3. The cells were seeded into a 5000 mL shake flask at a density of 2×10⁵ cells/mL, and collected by centrifugation on D3. The cells were seeded into a 50 L stirred reactor at a density of 2×10⁵ cells/mL, and collected by centrifugation on D3.

### S2: Expansion of Cells in a Reactor

The cells harvested from S1 were seeded into a 200 L stirred reactor containing a serum-free culture medium at a density of 2×10⁵ cells/mL. The stirring speed of the stirred reactor was set to 85 rpm, the pH was set to 7.2±0.2, the temperature was set to 37° C., and the minimum dissolved oxygen was set to 40%.

A serum-free feed culture medium was supplemented at 10% of the culture volume from D3 of culture, and the serum-free feed culture medium was supplemented at 10% of the culture volume every 2 days thereafter.

### S3: Differentiation of Cells in a Reactor

After 5 days of expansion of cells in a reactor, the minimum dissolved oxygen in the stirred reactor was adjusted to 10% to perform hypoxia-induced adipogenic differentiation of cells in suspension. The serum-free feed culture medium was continuously supplemented at 10% every 2 days, and differentiation of the cells in suspension was completed after 5 days of continuous culture.

During an expansion culture period of the cells in the reactor, the dissolved oxygen in the stirred reactor was tracked and detected. As shown in Table 3 and FIG. 5, the dissolved oxygen in the cell reactor gradually decreased from 95% to around 40% as the cells expanded. This was because the minimum dissolved oxygen was set at 40%, and the cell reactor would replenish oxygen to maintain the dissolved oxygen at around 40%. On D6, due to the minimum dissolved oxygen set at 10%, the dissolved oxygen in the cell reactor rapidly decreased to around 10%.

TABLE 3

| Days | D 0 | D 1 | D 2 | D 3 | D 4 | D 5 | D 6 | D 7 | D 8 | D 9 | D 10 |
|---|---|---|---|---|---|---|---|---|---|---|---|
| Dissolved oxygen (%) | 94.9 | 89.0 | 39.6 | 39 | 35.4 | 41.6 | 11.9 | 11.3 | 14.4 | 11.2 | 10.4 |

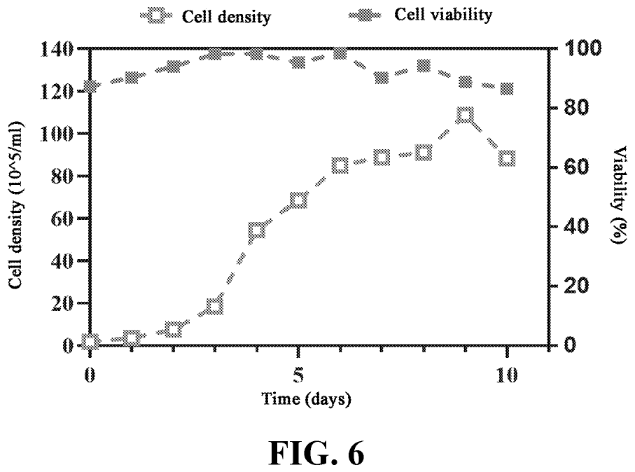

During an expansion culture period of the cells in the reactor, the corresponding growth curve was obtained by counting daily. The growth curve is shown in FIG. 6, and the cell growth condition was still in good condition during culture in the 200 L pilot scale reactor.

During a differentiation culture period of the cells in the reactor, the corresponding growth curve was obtained by counting daily, and oil red O staining detection was performed on lipid droplets on D5, D8, and D10. The growth curve is shown in FIG. 6. During the cell differentiation process, the number of cells could still be maintained, and the viability remained at 80% or higher all the time.

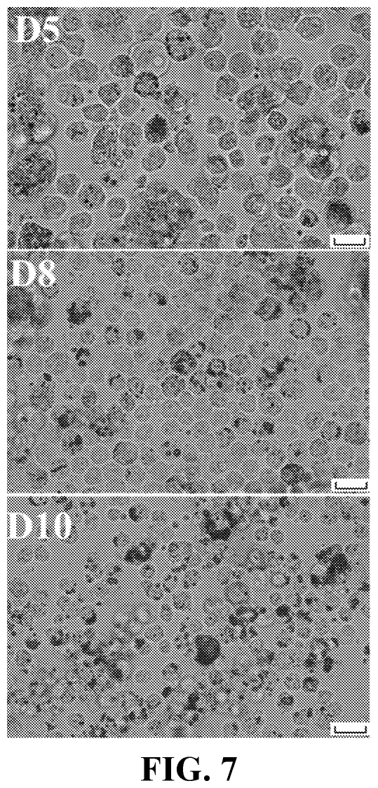

The oil red O staining results of the lipid droplets after hypoxia-induced cell differentiation are shown in FIG. 7. During the cell differentiation process from D5 to D10, the cells continuously accumulated the lipid droplets, and a large number of cells forming multilocular lipid droplets were observed on D10.

### Example 3

This example provides a safe and efficient method for hypoxia-induced adipogenic differentiation of cells, which is specifically a method for hypoxia-induced adipogenic differentiation of cells in suspension.

The method for hypoxia-induced adipogenic differentiation of cells in suspension, referring to Example 1, specifically includes the following steps:

### S1: Expansion of Cells in a Shake Flask

A stem cell line YP-S4-S-SC was thawed using a serum-free culture medium, and seeded into a 125 mL shake flask containing a serum-free culture medium at a density of 2×10⁵ cells/mL. The temperature was set to 37° C. and the rotation speed was set to 120 rpm. The cells were collected by centrifugation at 500×g for 5 min on D3. The cells were seeded into a 1000 mL shake flask at a density of 2×10⁵ cells/mL, and collected by centrifugation on D3.

### S2: Expansion of Cells in a Reactor

The cells harvested from S1 were seeded into a 50 L wave reactor containing a serum-free culture medium at a density of 2×10⁵ cells/mL. The angle of the wave reactor was set to 7°, the swing speed was set to 20 rpm, the pH was set to 7.2±0.2, the temperature was set to 37° C., and the minimum dissolved oxygen was set to 40%.

Perfusion culture was started at a rate of 0.5 volume to volume dilution (VVD) from D3 of culture, at a perfusion rate of 1 VVD on D4, and at a perfusion rate of 2 VVD on D5 and subsequent days.

### S3: Differentiation of Cells in a Reactor

After 5 days of expansion of cells in a reactor, the minimum dissolved oxygen in the reactor was adjusted to 10% to perform hypoxia-induced adipogenic differentiation of cells in suspension. Continuous perfusion culture was performed at 2 VVD during the period, and differentiation of the cells in suspension was completed after 5 days of continuous culture.

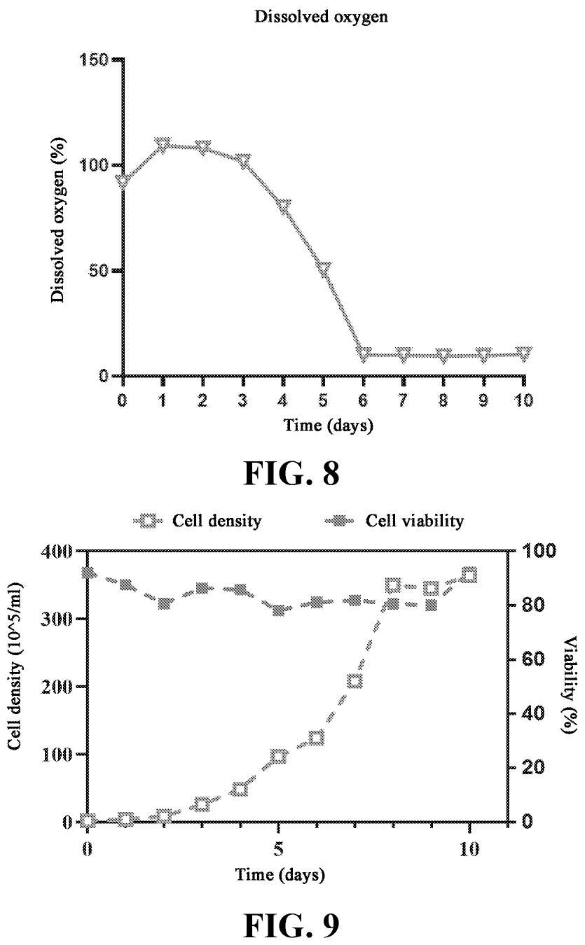

During an expansion culture period of the cells in the reactor, the dissolved oxygen in the reactor was tracked and detected. As shown in Table 4 and FIG. 8, the dissolved oxygen in the cell reactor increased firstly from 92% with cell expansion (culture in the wave reactor requires constant air flow, and the initial air flow is greater than the oxygen consumption of the cells, resulting in an increase in the dissolved oxygen level) and then decreased, and then decreased to around 50% on D5. On D6, due to the minimum dissolved oxygen set at 10%, the dissolved oxygen in the cell reactor rapidly decreased to around 10%.

TABLE 4

| Days | D 0 | D 1 | D 2 | D 3 | D 4 | D 5 | D 6 | D 7 | D 8 | D 9 | D 10 |
|---|---|---|---|---|---|---|---|---|---|---|---|
| Dissolved oxygen (%) | 91.8 | 109.4 | 108.2 | 101.9 | 80.3 | 50.6 | 10.1 | 10 | 9.6 | 9.8 | 10.4 |

During an expansion culture period of the cells in the reactor, the corresponding growth curve was obtained by counting daily. The growth curve is shown in FIG. 9, the cell growth condition is still in good condition during culture in the 50 L pilot scale reactor, and the cell density is slightly higher than the corresponding cell densities in Example 1 and Example 2.

During a differentiation culture period of the cells in the reactor, the corresponding growth curve was obtained by counting daily, and oil red O staining detection was performed on lipid droplets on D5 to D10. The growth curve is shown in FIG. 9. During the cell differentiation process, the number of cells could still be maintained, and the cell density was much higher than the corresponding cell densities in Example 1 and Example 2, indicating that the perfusion culture process could achieve a higher peak density than the fed-batch culture process, and the viability remained at 80% or higher all the time.

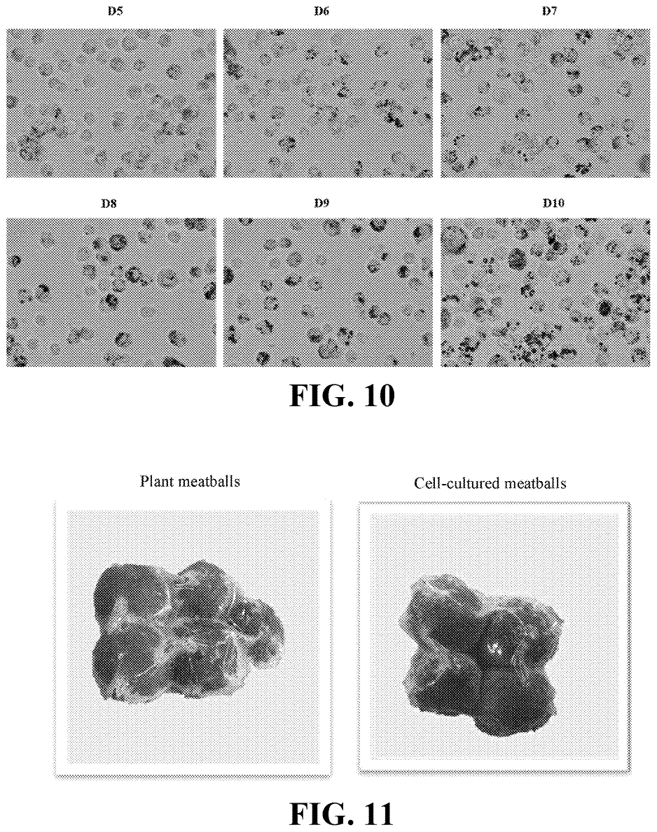

The oil red O staining results of the lipid droplets after hypoxia-induced cell differentiation are shown in FIG. 10. During the cell differentiation process from D5 to D10, the cells continuously accumulated the lipid droplets, and a large number of cells forming multilocular lipid droplets were observed on D10.

### Example 4

This example provides the safe and efficient method for hypoxia-induced adipogenic differentiation of cells of the present application, and use of cells obtained by hypoxia-induced adipogenic differentiation in preparation of cell-cultured meat.

In this example, the nutritional impact of addition of the cells on plant meatballs was mainly evaluated, including the following steps:

(1) Preparation of cell-cultured meat: A powder, ice water, and other auxiliary materials were mixed evenly, and shredded textured soy protein and the cells obtained by hypoxia-induced adipogenic differentiation accounting for 20% of the total weight of the final product (Example 2) were added. The mixture was kneaded into balls and fried at 170° C. for 1 min to set. After boiling in water, the center temperature of the meatballs reached 72° C., and then the meatballs were taken out and cooled in cold water to room temperature. The shaped meatball product was frozen at −20° C. for storage (FIG. 11).

TABLE 5
Nutritional composition of cell-cultured
meatballs and plant meatballs

| Indicator | Unit | Cell-cultured meatballs | Plant meatballs |
|---|---|---|---|
| Protein | g/100 g | 16.3 | 14.9 |
| Fat | g/100 g | 4.2 | 3.7 |
| Carbohydrate | g/100 g | 10.0 | 12 |
| Water content | g/100 g | 67.7 | 67.5 |
| Ash content | g/100 g | 1.8 | 1.9 |
| Calories | kJ/100 g | 602 | 594 |

(2) Detection of nutritional composition of cell-cultured meat: The shaped meatball products were taken out to thaw. The protein content was detected by the Kjeldahl method. The fat content was detected by the Soxhlet extraction method. The water content was detected by a drying method. The ash content was obtained by high-temperature incineration carbonization at 500° C., cooling, and weighing. The carbohydrate content was obtained by a subtraction method, namely subtracting the content of the protein, fat, water, and ash from the total weight. The calories were calculated by measuring the calories released by the main nutrients in the food, such as the protein, fat, and carbohydrate. The results are shown in Table 5. Compared with the plant meatballs without addition, the content of protein and fat in the cell-cultured meatballs with addition of the cell raw material at 20% were significantly increased, indicating that the addition of the cell raw material effectively improved the nutritional value of the plant meatballs, making the nutritional composition thereof closer to that of a traditional meat product.

What is claimed is:

1. A method for hypoxia-induced adipogenic differentiation of cells, comprising the following steps:
S1: culturing cells using a culture medium for cell expansion, with dissolved oxygen controlled at no less than 35% during the culture process; and
S2: after the cell expansion, controlling the dissolved oxygen at no greater than 20% and continuing culturing to induce adipogenic differentiation of the cells.

2. The method for hypoxia-induced adipogenic differentiation of cells according to claim 1, wherein
in S1, the dissolved oxygen at an initial stage of culture is controlled at 90-100%; and
in S2, the dissolved oxygen is controlled at 1-20%.

3. The method for hypoxia-induced adipogenic differentiation of cells according to claim 2, wherein
in S2, the dissolved oxygen is controlled at 9-20%.

4. The method for hypoxia-induced adipogenic differentiation of cells according to claim 2, wherein
in S1, the culture time is 3-10 days;
in S2, the culture time is 3-10 days.

5. The method for hypoxia-induced adipogenic differentiation of cells according to claim 4, wherein
the cells are a pig muscle stem cell line adapted to carrier-free and serum-free suspension culture;
the culture medium is a serum-free culture medium.

6. The method for hypoxia-induced adipogenic differentiation of cells according to claim 5, wherein
the culture process is a fed-batch culture process; or
the culture process is a perfusion culture process.

7. The method for hypoxia-induced adipogenic differentiation of cells according to claim 6, wherein
the culture process is the fed-batch culture process, and the S1 comprises:
seeding cells harvested from cell thawing into a stirred bioreactor containing a serum-free culture medium at a density of (1-10)×10⁵ cells/mL, setting a stirring speed to 80-240 rpm, a pH value to (6.0-8.0)±0.2, and a temperature to 34-40° C., setting a minimum dissolved oxygen, supplementing a serum-free feed culture medium from D3 of culture, and supplementing the feed culture medium every 2 days thereafter.

8. The method for hypoxia-induced adipogenic differentiation of cells according to claim 6, wherein
the culture process is the perfusion culture process, and the S1 comprises:
seeding cells harvested from cell thawing into a wave bioreactor containing a serum-free culture medium at a density of (1-10)×10⁵ cells/mL, setting an angle of the wave bioreactor to 6-9°, a swing speed to 15-30 rpm, a pH value to (7.0-7.6)±0.2, and a temperature to 34-40° C., setting a minimum dissolved oxygen, and starting perfusion culture at a rate of 0.5 VVD from D3 of culture, then at a rate of 1 VVD on D4, and at a rate of 2 VVD from D5.

9. The method for hypoxia-induced adipogenic differentiation of cells according to claim 7, wherein the cell thawing comprises:
seeding cells in a serum-free culture medium at a density of (1-10)×10⁵ cells/mL, collecting the cells by centrifugation every 2-6 days for passage, repeating to obtain a sufficient number of cells, and harvesting the cells by centrifugation.

10. The method for hypoxia-induced adipogenic differentiation of cells according to claim 8, wherein the cell thawing comprises:
seeding cells in a serum-free culture medium at a density of (1-10)×10⁵ cells/mL, collecting the cells by centrifugation every 2-6 days for passage, repeating to obtain a sufficient number of cells, and harvesting the cells by centrifugation.

11. Use of the method for hypoxia-induced adipogenic differentiation of cells according to claim 1 in preparation of cell-cultured meat.
